(19) United States
(12) Patent Application Publication
(10) Pub. No.: US 2014/0161896 A1
(43) Pub. Date: Jun. 12, 2014

(54) PARTICLES FOR THE TREATMENT OF NEURODEGENERATIVE DISEASES

(71) Applicants: Dan Peer, Kiryat-Ono (IL); Mia Horowitz, Ramat-HaSharon (IL)

(72) Inventors: Dan Peer, Kiryat-Ono (IL); Mia Horowitz, Ramat-HaSharon (IL)

(73) Assignee: Ramot at Tel-Aviv University Ltd., Tel-Aviv (IL)

(21) Appl. No.: 14/236,909

(22) PCT Filed: Jul. 31, 2012
(86) PCT No.: PCT/IL2012/050281

§ 371 Date: Feb. 4, 2014

Related U.S. Application Data
(60) Provisional application No. 61/514,925, filed on Aug. 4, 2011.

(51) Int. Cl. A61K 38/47; A61K 9/51; A61K 45/06; A61K 9/00; A61K 31/137

(52) U.S. Cl. CPC A61K 38/47; A61K 9/0043; A61K 31/137; A61K 45/06; A61K 9/5161; A61K 9/5123
USPC 424/499; 424/94.61

(57) ABSTRACT

A composition of matter comprising non-cellular particles, which comprise a lysosomal enzyme and/or a small molecule which increases an amount and/or activity of a lysosomal enzyme.

### FIELD AND BACKGROUND OF THE INVENTION

The present invention, in some embodiments thereof, relates to a method of treating neurodegenerative diseases, by administration of particles which comprise agents that upregulate an amount or activity of lysosomal enzymes in lysosomes of brain cells.

Parkinson's disease (PD) is an age-related disorder characterized by progressive loss of dopamine producing neurons in the substantia nigra of the midbrain, which in turn leads to progressive loss of motor functions manifested through symptoms such as tremor, rigidity and ataxia. Parkinson's disease can be treated by administration of pharmacological doses of the precursor of dopamine, L-DOPA (Marsden, Trends Neurosci. 9:512, 1986; Vinken et al., in Handbook of Clinical Neurology p. 185, Elsevier, Amsterdam, 1986). Although such treatment is effective in early stage Parkinson's patients, progressive loss of substantia nigra cells eventually leads to an inability of remaining cells to synthesize sufficient dopamine from the administered precursor and to diminishing pharmacogenic effect.

Recently mutations of the lysosomal enzyme glucocerebrosidase (GCase) were found to represent a significant risk factor for the development of Parkinson's disease (PD) and it has been suggested that this is the most common genetic factor identified for PD to date (see for example Aharon-Peretz et al., N Engl J Med. 2004 Nov. 4;351(19):1972-7).

Delivery of GCase to the brain has been suggested for the treatment of patients with neuronopathic Gaucher disease and other neurological disorders (Lonser et al., Neurology Jan. 23, 2007 vol. 68 no. 4 254-261).

Zhang et al (Pharmaceutical Research, Volume 25, Number 2, P.400-406, 2008) teaches administering particles, which comprise polynucleotides encoding B-glucoronidase for the treatment of type VII mucopolysaccharidosis, wherein the particles are targeted across the blood brain barrier using a monoclonal antibody to the mouse transferrin receptor.

U.S. Patent Application No. 20090155178 teaches lipidated glycosaminoglycans which encapsulate drugs for subsequent delivery for use in therapy and diagnosis.

### SUMMARY OF THE INVENTION

According to an aspect of some embodiments of the present invention there is provided a composition comprising particles which encapsulate an agent selected from the group consisting of lysosomal enzyme, a small molecule which lowers an amount of a substrate of a lysosomal enzyme in a lysosome of a cell and a combination thereof.

According to an aspect of some embodiments of the present invention there is provided a method of treating a neurodegenerative disorder, comprising administering to a subject in need thereof a therapeutically effective amount of an agent selected from the group consisting of a lysosomal enzyme, a small molecule which lowers an amount of a substrate of a lysosomal enzyme in brain cells of the subject and a combination thereof, wherein the lysosomal enzyme and the small molecule are encapsulated within particles, thereby treating the neurodegenerative disorder.

According to an aspect of some embodiments of the present invention there is provided a pharmaceutical composition comprising the composition described herein.

According to some embodiments of the invention, the particles are nanoparticles.

According to some embodiments of the invention, the administering is systemically administering.

According to some embodiments of the invention, the systemically administering is selected from the group consisting of intravenous (IV), intra-arterial (IA), intramuscular (M), subcutaneous (SC), intraperitoneal (IP), intracranial and intranasal.

According to some embodiments of the invention, the administering comprises intranasally administering.

According to some embodiments of the invention, the particles are selected from the group consisting of polymeric particles, microcapsules, liposomes, microspheres, microemulsions, nanoparticles, nanocapsules, nanospheres and nanocages.

According to some embodiments of the invention, the particles have a charged external surface.

According to some embodiments of the invention, the particles comprise a neutral external surface.

According to some embodiments of the invention, the particles comprise lipids.

According to some embodiments of the invention, the lipids comprise cationic lipids.

According to some embodiments of the invention, the cationic lipid is selected from the group consisting of 1,2-Dilauroyl-sn-Glicero-3-Phosphoethanolamine (DLPE) and 1,2-Dilauroyl-sn-Glicero-3-Glycerol (DLPG), dioleoyl-1,2-diacyl-3-trimethylammonium-propane (DOTAP, at 18:1; 14:0; 16:0, 18:0) and N-[1-(2,3-dioleyloxy)propyl]-N,N,N-trimethlylammonium chloride (DOTMA); dimethyldioctadecylammonium (DDAB); 1,2-dilauroyl-sn-glycero-3-ethylphosphocholine (Ethyl PC, at 12:0; 14:0; 16:0; 18:0; 18:1; 16:0-18:1); 1,2-di-(9Z-octadecenoyl)-3-dimethylammonium-propane and 3B- [N-(N′,N′-dimethylaminoethane)-carbamoyl]cholesterol hydrochloride (DC-Cholesterol).

According to some embodiments of the invention, the lipids comprise a neutral lipid.

According to some embodiments of the invention, the neutral lipid comprises phosphatidylethanolamine or dioleilphosphatidylethanolamine (DOPE).

According to some embodiments of the invention, the lipids comprise anionic phospholipids.

According to some embodiments of the invention, the anionic phospholipids are selected from the group consisting of phosphatidylserine, phosphatidic acid, phosphatidylcholine and phosphatidyl glycerol.

According to some embodiments of the invention, a targeting moiety is attached to an outer surface of the particles.

According to some embodiments of the invention, the targeting moiety is selected from the group consisting of an antibody, an antibody fragment, an aptamer and a receptor ligand.

According to some embodiments of the invention, the targeting moiety comprises a glycosaminoglycan.

According to some embodiments of the invention, the glycosaminoglycan is selected from the group consisting of hyaluronic acid (HA), keratan sulfate, chondroitin sulfate, heparin sulfate, heparan sulfate, dermatin sulfate, salts, and mixtures thereof.

According to some embodiments of the invention, the glycosaminoglycan comprises HA.

According to some embodiments of the invention, the neurodegenerative disorder is selected from the group consisting of Parkinson's, multiple sclerosis, epilepsy, amyotrophic lateral sclerosis, stroke, autoimmune encephalomyelitis, diabetic neuropathy, glaucomatous neuropathy, Alzheimer's disease and Huntingdon's disease.

According to some embodiments of the invention, the neurodegenerative disorder is Parkinson's.

According to some embodiments of the invention, the neurodegenerative disorder comprises a neurometabolic disorder.

According to some embodiments of the invention, the neurometabolic disorder comprises a lysosomal storage disease.

According to some embodiments of the invention, the lysosomal enzyme is selected from the group consisting of glucocerebrosidase (GCase), acid sphingomyelinase, hexosaminidase, α-N-acetylgalactosaminidise, acid lipase, α-galactosidase, α-L-iduronidase, iduronate sulfatase, α-mannosidase, sialidase, α fucosidase, G_{M1}-β- galctosidase, ceramide lactosidase, arylsulfatase A, β galactosidase and ceramidase.

According to some embodiments of the invention, the lysosomal enzyme is GCase.

According to some embodiments of the invention, the lysosomal enzyme comprises a recombinant lysosomal enzyme.

According to some embodiments of the invention, the small molecule increases an activity and/or amount of a lysosomal enzyme in brain cells of the subject.

According to some embodiments of the invention, the small molecule enhances a passage of a mutant lysosomal enzyme from the endoplasmic reticulum to the lysosome of brain cells of the subject.

According to some embodiments of the invention, the small molecule is co-formulated in the particles which comprise the lysosomal enzyme.

According to some embodiments of the invention, the small molecule is comprised in particles which do not comprise the lysosomal enzyme.

According to some embodiments of the invention, the lysosomal enzyme is GCase and the small molecule binds GCase.

According to some embodiments of the invention, the lysosomal enzyme is Gcase and the small molecule comprises a glucosyl-ceramide synthase inhibitor.

According to some embodiments of the invention, the lysosomal enzyme is GCase and the small molecule is Ambroxol.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### DESCRIPTION OF SPECIFIC EMBODIMENTS OF THE INVENTION

The present invention, in some embodiments thereof, relates to a method of treating neurodegenerative diseases, by administration of particles which comprise agents that upregulate an amount or activity or lysosomal enzymes in lysosomes of brain cells. Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Gaucher disease (GD), a sphingolipidosis characterized by impaired activity of the lysosomal enzyme glucocerebrosidase (GCase), results mainly from mutations in the GCase gene. The mutant GCase variants undergo ER Associated Degradation, a process which may be deleterious to cells, mainly brain cells. Recently GCase mutations were found to represent a significant risk factor for the development of Parkinson's disease (PD) and it is suggested that this is the most common genetic factor identified for PD to date.

GD was the first metabolic and lysosomal disease for which enzyme replacement therapy (ERT) was developed. The enzyme does not cross the blood brain barrier (BBB) and therefore does not ameliorate any neurological signs. Its ability to reach other cells has never been tested since the affected cells in GD are macrophages.

The present inventors propose the use of particles carrying a lysosomal enzyme GCase) or a small molecule agent which reduces the amount of substrate for the lysosomal enzyme, or a combination of both, with the ability to reach the brain and deliver its contents to cells therein for the treatment of neurodegenerative diseases such as Parkinsons.

Thus, according to one aspect of the present invention, there is provided a composition of matter comprising particles which encapsulate a lysosomal enzyme and/or a small molecule which lowers an amount of a substrate of a lysosomal enzyme in a lysosome of a cell.

As used herein, “particles” refers to structures which are not biological cells.

The particle may be a synthetic carrier, gel or other object or material having an external surface which is capable of encapsulating an agent. The particle may be either polymeric or non-polymeric preparations.

Exemplary particles that may be used according to this aspect of the present invention include, but are not limited to polymeric particles, microcapsules, liposomes, microspheres, microemulsions, nanoparticles, nanocapsules, nano-spheres, nano-liposomes, nano-emulsions and nanotubes.

According to a particular embodiment, the particles are nanoparticles.

As used herein, the term “nanoparticle” refers to a particle or particles having an intermediate size between individual atoms and macroscopic bulk solids. Generally, nanoparticle has a characteristic size (e.g., diameter for generally spherical nanoparticles, or length for generally elongated nanoparticles) in the sub-micrometer range, e.g., from about 1 nm to about 500 nm, or from about 1 nm to about 200 nm, or of the order of 10 nm, e.g., from about 1 nm to about 100 nm. The nanoparticles may be of any shape, including, without limitation, elongated particle shapes, such as nanowires, or irregular shapes, in addition to more regular shapes, such as generally spherical, hexagonal and cubic nanoparticles. According to one embodiment, the nanoparticles are generally spherical.

The particles of this aspect of the present invention may have a charged surface (i.e., positively charged or negatively charged) or a neutral surface.

Agents which are used to fabricate the particles may be selected according to the desired charge required on the outer surface of the particles.

Thus, for example if a negatively charged surface is desired, the particles may be fabricated from negatively charged lipids (i.e. anionic phospholipids) such as described herein below.

When a positively charged surface is desired, the particles may be fabricated from positively charged lipids (i.e. cationic phospholipids), such as described herein below.

As mentioned, non charged particles are also contemplated by the present invention. Such particles may be fabricated from neutral lipids such as phosphatidylethanolamine or dioleilphosphatidylethanolamine (DOPE).

It will be appreciated that combinations of different lipids may be used to fabricate the particles of the present invention, including a mixture of more than one cationic lipid, a mixture of more than one anionic lipid, a mixture of more than one neutral lipid, a mixture of at least one cationic lipid and at least one anionic lipid, a mixture of at least one cationic lipid and at least one neutral lipid, a mixture of at least one anionic lipid and at least one neutral lipid and additional combinations of the above.

There are numerous polymers which may be attached to lipids. Polymers typically used as lipid modifiers include, without being limited thereto: polyethylene glycol (PEG), polysialic acid, polylactic (also termed polylactide), polyglycolic acid (also termed polyglycolide), apolylactie- polyglycolic acid' polyvinyl alcohol, polyvinylpyrrolidone, polymethoxazoline, polyethyloxazoline, polyllydroxyetlyloxazolille, solyhydroxypryloxazoline, polyaspartarllide, polyhydroxypropyl methacrylamide, polymethacrylamide, polydimethylacrylamide, polyvinylmethylether, polyhydroxyethyl acrylate, derivatized celluloses such as hydroxymethylcellulose or hydroxyethylcellulose.

The polymers may be employed as homopolymers or as block or random copolymers.

The particles may also include other components. Examples of such other components includes, without being limited thereto, fatty alcohols, fatty acids, and/or cholesterol esters or any other pharmaceutically acceptable excipients which may affect the surface charge, the membrane fluidity and assist in the incorporation of the biologically active lipid into the lipid assembly. Examples of sterols include cholesterol, cholesterol hemisuccinate, cholesterol sulfate, or any other derivatives of cholesterol. Preferred lipid assemblies according the invention include either those which form a micelle (typically when the assembly is absent from a lipid matrix) or those which form a liposome (typically, when a lipid matrix is present).

In a specific embodiment, the particle is a liposome. As used herein and as recognized in the art, liposomes include any synthetic (i.e., not naturally occurring) structure composed of lipid bilayers, which enclose a volume. Liposomes include emulsions, foams, micelles, insoluble monolayers, liquid crystals, phospholipid dispersions, lamellar layers and the like. The liposomes may be prepared by any of the known methods in the art [Monkkonen, J. et al., 1994, J. Drug Target, 2:299-308; Monkkonen, J. et al., 1993, Calcif. Tissue Int., 53:139-145; Lasic D D., Liposomes Technology Inc., Elsevier, 1993, 63-105. (chapter 3); Winterhalter M, Lasic D D, Chem Phys Lipids, 1993 Sep.;64(1-3):35-43].

The liposomes may be unilamellar or may be multilamellar. Unilamellar liposomes may be preferred in some instances as they represent a larger surface area per lipid mass. Suitable liposomes in accordance with the invention are preferably non-toxic. The liposomes may be fabricated from a single phospholipid or mixtures of phospholipids. The liposomes may also comprise other lipid materials such as cholesterol. For fabricating liposomes with a negative electrical surface potential, acidic phospho- or sphingo- or other synthetic-lipids may be used. Preferably, the lipids have a high partition coefficient into lipid bilayers and a low desorption rate from the lipid assembly. Exemplary phospholipids that may be used for fabricating liposomes with a negative electrical surface potential include, but are not limited to phosphatidylserine, phosphatidic acid, phosphatidylcholine and phosphatidyl glycerol.

Other negatively charged lipids which are not liposome forming lipids that may be used are sphingolipids such as cerebroside sulfate, and various gangliosides. The most commonly used and commercially available lipids derivatized into lipopolymers are those based on phosphatidyl ethanolamine (PE), usually distearylphosphatidylethanolamine (DSPE).

The lipid phase of the liposome may comprise a physiologically acceptable liposome forming lipid or a combination of physiologically acceptable liposome forming lipids for medical or veterinarian applications. Liposome-forming lipids are typically those having a glycerol backbone wherein at least one of the hydrofoil groups is substituted with an acyl chain, a phosphate group, a combination or derivatives of same and may contain a chemically reactive group (such as an as amine imine, acids ester, aldelhyde or alcohol) at the headgroup. Typically, the acyl chain is between 12 to about 24 carbon atoms in length, and has varying degrees of saturation being fully, partially or non-hydrogenated lipids. Further, the lipid matrix may be of natural source, semi-synthetic or fully synthetic lipid, and neutral, negatively or positively charged.

According to one embodiment, the lipid phase comprises phospholipids. The phospholipids may be a glycerophospholipid. Examples of glycerophospholipid include, without being limited thereto, phosphatidylglycerol (PG) including dimyristoyl phosphatidylglycerol (DMPG); phosphatidylcholine (PC), including egg yolk phosphatidylcholine and dimyristoyl phosphatidylcholine (DMPC), phosphatidic acid (PA), phosphatidylinositol (PI), phosphatidylserine (PS) and sphingomyelin (SM) and derivatives of the same.

Another group of lipid matrix employed according to the invention includes cationic lipids (monocationic or polycationic lipids). Cationic lipids typically consist of a lipophilic moiety, such as a sterol or the same glycerol backbone to which two acyl or two alkyl, or one acyl and one alkyl chain contribute the hydrophobic region of the amphipathic molecule, to form a lipid having an overall net positive charge.

Preferably, the head groups of the lipid carries the positive charge. Monocationic lipids may include, for example, 1,2-dimyristoyl-3- trimethylammonium propane (DMTAP) 1,2-dioleyloxy-3-(trimethylanino) propane (DOTAP), N-[-1-(2,3,-ditetradecyloxy)propyl]-N,N-dimethyl-N-hydroxyethylammonium bromide (DMRIE), N-[1-(2,3,- dioleyloxy)propyl]-N,N- dimethyl-N-hydroxy ethyl-ammonium bromide (DORIE), N-[1-(2,3-dioleyloxy) propyl];-N,N,N-trimethylammonium chloride (DOTMA); 3;N-(N′,N′- dimethylaminoethane) carbamoly]; cholesterol (DC-Chol), and I dimethyl-dioctadecylammonium (DDAB).

Examples of polycationic lipids include a similar lipoplilic moiety as with the mono cationic lipids, to which spermine or spermidine is attached. These include' without being limited thereto, N-[2-[[2,5-bis[3-aminopropyl)amino]-1-oxopentyl]amino]ethyl]N,N dimethul-2,3 bis (1-oXo-9-octadecenyl) oXy];-1 propanaminium (DOSPA), and ceramide carbamoyl spermine (CCS).

The cationic lipids may be used alone, in combination with cholesterol, with neutral phospholipids or other known lipid assembly components. In addition, the cationic lipids may form part of a derivatized phospholipids such as the neutral lipid dioleoylphosphatidyl ethanolamine (DOPE) derivatized with polylysine to form a cationic lipopolymer.

The diameter of the liposomes used preferably ranges from 50-200 nM and more preferably from 20-100 nM. For sizing liposomes, extrusion, homogenization or exposure to ultrasound irradiation may be used, Homogenizers which may be conveniently used include microfluidizers produced by Microfluidics of Boston, Mass. In a typical homogenization procedure, liposomes are recirculated through a standard emulsion homogenizer until selected liposomes sizes are observed. The particle size distribution can be monitored by conventional laser beam particle size discrimination.

Extrusion of liposomes through a small-pore polycarbonate membrane or an asymmetric ceramic membrane is an effective method for reducing liposome sizes to a relatively well defined size distribution. Typically, the suspension is cycled through the membrane one or more times until the desired liposome size distribution is achieved. The liposomes may be extruded through successively smaller pore membranes to achieve a gradual reduction in liposome size.

According to another embodiment, the particle is a nanoparticle. Preferably, nanoparticles are less than 100 nm in diameter and can be spherical, non-spherical, or polymeric particles. In a preferred embodiment, the polymer used for fabricating nanoparticles is biocompatible and biodegradable, such as poly(DL-lactide-co-glycolide) polymer (PLGA). However, additional polymers which may be used for fabricating the nanoparticles include, but are not limited to, PLA (polylactic acid), and their copolymers, polyanhydrides, polyalkyl-cyanoacrylates (such as polyisobutylcyanoacrylate), polyethyleneglycols, polyethyleneoxides and their derivatives, chitosan, albumin, gelatin and the like.

The particles of the present invention may be modified. According modified to enhance their circulatory half-life (e.g. by PEGylation) to reduce their clearance and prolong their scavenging time-frame. The PEG which is incorporated into the articles may be characterized by of any of various combinations of chemical composition and/or molecular weight, depending on the application and purpose.

According to one embodiment, selection of the formulation of the particle will be effected so as to promote crossing of the blood brain barrier.

Another exemplary modification of the particles of the present invention is attachment of a targeting moiety to bind cell surface markers or to enhance the crossing of the blood brain barrier.

As used herein, the phrase “surface marker”, refers to any chemical structure which is specifically displayed, displayed at uniquely high density, and/or displayed in a unique configuration by a cell surface or extracellular matrix of the target cell/tissue.

For the purposes of the present disclosure, the term “targeting moiety” refers to any ligand or ligand receptor which can be incorporated into complexes. Such ligands can include, but are not limited to, antibodies such as IgM, IgG, IgA, IgD, and the like, or any portions or subsets thereof, cell factors, cell surface receptors such as, integrins, proteoglycans, sialic acid residues, etc., and ligands therefore, MHC or HLA markers, viral envelope proteins, peptides or small organic ligands, derivatives thereof, and the like.

Of particular interest for targeted gene delivery applications are proteins encoding various cell surface markers and receptors. A brief list that is exemplary of such proteins includes, but is not limited to: CD1(a-c), CD4, CD8-11(a-c), CD15, CDw17, CD18, CD21-25, CD27, CD30-45(R(O, A, and B)), CD46-48, CDw49(b,d,f), CDw50, CD51, CD53-54, CDw60, CD61-64, CDw65, CD66-69, CDw70CD71, CD73-74, CDw75, CD76-77, LAMP-1 and LAMP-2, and the T-cell receptor, integrin receptors, endoglin for proliferative endothelium, or antibodies against the same.

According to a specific embodiment, the targeting moiety is a glycosaminoglycan, including, but not limited to hyaluronic acid (HA), keratan sulfate, chondroitin sulfate, heparin sulfate, heparan sulfate, dermatin sulfate, salts, and mixtures thereof.

According to another embodiment the particle is one which is described in WO2001/013100 and U.S. Pat. Nos. 7,544,374 and 8,178,120, each of which are incorporated herein by reference.

It has been suggested that the use of an external ligand such as mannose can improve a liposomal particle's ability to cross the BBB [Huitinga et al., J exp Med 172 (1990) 1025-33; Umezawa F., Biochem Biophys Res Commun 153 (1988) 1038-44]. PCT Application, Publication No. WO9402178A1 to Micklus, incorporated herein by reference discusses the coupling of liposomes to an antibody binding fragment which binds to a receptor molecule present on the vascular endothelial cells of the mammalian blood-brain barrier.

As mentioned, the particles of the present invention encapsulate at least one active agent—e.g. a lysosomal enzyme and/or a small molecule agent which lowers an amount of a substrate of a lysosomal enzyme.

The term “encapsulated” as used herein refers to the agent being distributed in the interior portion of the particles. Preferably, the active agents are homogenously distributed. Homogeneous distribution of an active agent in polymer particles is known as a matrix encapsulation. However, due to the manufacturing process it is foreseen that minor amounts of the active agent may also be present on the outside of the particle and/or mixed with the polymer making up the shell of the particle.

The particles comprising the active agents should be formulated to sequester the active agents for a sufficient time to allow for delivery of the agent to the brain. In the case where a more stable liposome formulation is required, incorporation of a certain amount of cholesterol in the particle results in a decrease of their intracellular degradation by macrophages. It has also been shown that the addition of cholesterol to a liposome formulation increases the sequestering efficiency of the liposome by about two fold [Mumper et al., AAPS PharmSciTech, 2000;1 (1) article 3].

As used herein, the phrase “lysosomal enzyme” refers to acid hydrolases typically found in the lysosomes of the cell. The lysosomal enzyme may be a nuclease, a protease, a glycosidase, a lipase, a phosphatase, a sulfatase or a phospholipase. Exemplary lysosomal enzymes include, but are not limited to glucocerebrosidase (GCD), acid sphingomyelinase, hexosaminidase, α-N-acetylgalactosaminidise, acid lipase, α-galactosidase, α-L-iduronidase, iduronate sulfatase, α-mannosidase, sialidase, α fucosidase, G_{M1}-β- galctosidase, ceramide lactosidase, arylsulfatase A, β galactosidase and ceramidase.

The EC numbers of exemplary lysosomal enzymes are listed in Table 1 below. A lysosomal enzyme of the present invention also refers to homologs and other modifications including additions or deletions of specific amino acids to the sequence (e.g., polypeptides which are at least 50%, at least 55%, at least 60%, at least 65%, at least 70%, at least 75%, at least 80%, at least 85%, at least 87%, at least 89%, at least 91%, at least 93%, at least 95% or more say 100% homologous to the lysomal amino acid sequences listed in Table 1 as determined using BlastP software of the National Center of Biotechnology Information (NCBI) using default parameters). The homolog may also refer to a deletion, insertion, or substitution variant, including an amino acid substitution, thereof and biologically active polypeptide fragments thereof.

TABLE 1

| Lysosomal enzyme | EC Number |
|---|---|
| glucocerebrosidase (GCD) | EC 3.2.1.45 |
| Sphingomyelinase | E.C. 3.1.4.12 |
| Hexosaminidase | E.C. 3.2.1.52 |
| α-galactosidase | E.C. 3.2.1.22 |
| α-L-iduronidase | E.C. 3.2.1.76 |
| iduronate sulfatase | E.C. 3.1.6.13 |
| α-mannosidase | E.C. 3.2.1.113 |
| Sialidase | E.C. 3.2.1.18 |
| α fucosidase | EC 3.2.1.63 |
| arylsulfatase A | E.C. 3.1.6.8 |
| β galactosidase | EC 3.2.1.23 |
| Ceramidase | EC 3.5.1.23 |
| β-glucoronidase | E.C.3.2.1.31 |

Lysosomal enzymes may be isolated from tissues such as the placenta (e.g. β-glucocerebrosidase can be prepared from placenta as Ceredase™).

Methods of generating recombinant lysosomal enzymes are known in the art. For example, Radin et al., U.S. Pat. No. 5,929,304 teach plant systems for expression of lysosomal enzymes in general. Recombinant α-Galactosidase A has been produced in insect (sf9) cells (see U.S. Pat. No. 7,011,831) in human fibroblasts (see U.S. Pat. No. 6,395,884) and in plant cells (see U.S. Pat. No. 6,846,968).

According to one embodiment, the lysosomal enzymes of the present invention comprise a human amino acid sequence.

According to another embodiment, the lysosomal enzymes are plant lysosomal enzymes.

According to a particular embodiment of this aspect of the present invention the lysosomal enzyme (e.g. a-Galactosidase A [a-GAL] or glucocerebrosidase [GCD]) comprises a human amino acid sequence and is recombinantly generated in a plant.

Alternatively, the lysosomal enzyme may be recombinantly generated in mammalian cells (or isolated from mammalian cells) and moieties which increase cellular uptake of the liposomal enzyme are modified. For example, U.S. Pat. No. 20090041741 teaches a modified recombinant B-glucuronidase wherein its carbohydrate moieties are chemically modified so as to reduce its activity with respect to mannose and mannose 6-phosphate cellular delivery system while retaining enzymatic activity.

As mentioned, the particles of the present invention may also comprise small molecule agents which lowers an amount of a substrate of a lysosomal enzyme.

The substrate can be, but is not limited to, dermatan sulfate (metabolized by .alpha.-L-iduronidase, iduronate-2- sulfatase, galactosamine-4- sulfatase and/or .beta.-glucuronidase); heparin sulfate (metabolized by .alpha.-L-iduronidase, iduronate-2-sulfatase, heparan sulfamidase, N-acetyl-.alpha.-glucosaminidase, acetyl CoA glucosamine-N-acetyltransferase, N-acetylglucosamine-1-phosphotransferase, N-acetylglucosamine-6-sulfate sulfatase and/or .beta.-glucuronidase); keratan sulfate (metabolized by N-acetylgalactosamine-6-sulfate sulfatase and/or .beta.-galactosidase); hyaluronic acid (metabolized by hyaluronidase); sialic acid (metabolized by neuraminidase and/or sialic acid transporter); GM.sub.1-ganglioside (metabolized by .beta.-galactosidase); GM2-ganglioside (metabolized by .beta.-hexosaminidase A, 13-hexosaminidase B, GM.sub.2 activator); galactosylceramide (metabolized by galactosylceramidase); sulfatide (metabolized by arylsulfatase A and B); galactosylsphingolipids (metabolized by .alpha.-galactosidase A); glucoceramide (metabolized by .beta.-glucosidase), ceramide (metabolized by ceramidase); sphingomyelin (metabolized by sphingomyelinase); .alpha.-mannoside (metabolized by .alpha.-mannosidase); .beta.-mannoside (metabolized by .beta.-mannosidase); fucoside (metabolized by fucosidase); N-acetyl-.beta.-glucosamine (metabolized by N-acetyl-.beta.-glucosaminidase); N-acetylgalactosamine (metabolized by .alpha.-galactosidase, .alpha.-N-acetylgalactosaminidase); glycogen (metabolized by .alpha.-glucosidase); cholesterol ester (metabolized by acid lipase); bone-derived peptides (metabolized by cathepsin K); galactosialic acid (metabolized by cathepsin A); and saposins (metabolized by palmitoyl protein thioesterase).

Such agents include those that increase an activity and/or amount of a lysosomal enzyme in brain cells of the subject, those that enhance a passage of a mutant lysosomal enzyme from the endoplasmic reticulum to the lysosome (e.g. ambroxol) or those that inhibit formation of the substrate—e.g. in the case of Gcase, the small molecule agent may comprise a glucosyl-ceramide synthase inhibitor, including for example (±)-threo-1-Phenyl-2-decanoylamino-3-morpholino-1-propanol hydrochloride which is commercially available from Merck.

Glucosylceramide synthase is the enzyme catalyzing the first glycosylation step in the synthesis of glucosylceramide-based glycosphingolipids. Inhibitors thereof have two identified sites of action: the inhibition of glucosylceramide synthase, resulting in the depletion of cellular glycosphingolipids, and the inhibition of 1-O-acylceramide synthase, resulting in the elevation of cell ceramide levels.

As mentioned, the particles of the present invention may be used to treat brain diseases, such as neurodegenerative diseases and neurometabolic diseases e.g. lysosomal storage diseases.

Thus, according to another aspect of the present invention there is provided a method of treating a neurodegenerative disorder, comprising administering to a brain of the subject in need thereof a therapeutically effective amount of a lysosomal enzyme and/or a small molecule agent which lowers an amount of a substrate of a lysosomal enzyme in brain cells of the subject, wherein said lysosomal enzyme and said small molecule agent are encapsulated within non-cellular particles, thereby treating the neurodegenerative disorder.

Subjects which may be treated according to the methods described herein are typically mammalian subjects, e.g. human.

Examples of lysosomal storage diseases (and their associated mutant enzymes) include but are not limited to Fabry disease (a-galactosidase); Pompe Disease (acid α-1,4 glucosidase; acid α-1,6 glucosidase), GM1 gangliosidosis (β-galactosidase, Tay-Sachs disease (β-hexasaminidase A), GM2 gangliosialidosis ((β-hexasaminidase A), AB Variant and GM2 (mutant GM2 Activator Protein), Sandhoff Disease -(and β-hexosaminidase B), Gaucher Disease (glucocerebrosidase or saposin C of the prosaposin), Krabbe Disease (galactosylcerebrosidase), Niemann-Pick Type and B (acid sphingomyelinase), Farber Disease (acid ceramidase), Wolman Disease (acid lipase), Cholesterol Ester Storage Disease (acid lipase), Hurler Syndrome (α-L-iduronidase), Scheie Syndrome (α-L-iduronidase), Hurler-Scheie and α-L-iduronidase, Hunter Syndrome (iduronate 2-sulfatase), Sanfilippo A (α-N-acetylglucosaminidase), Sanfilippo B (α-N-acetylglucosaminidase), Sanfilippo C (acetyl-CoA-glucosaminide acetyltransferase), Sanfilippo D (N-acetylglucosamine-6-sulfatase), Morquio A (N-acetylglucosamine-6-sulfate sulfatase), Morquio B (β-galactosidase), Maroteaux-Lamy (arysuylfatase B), Metachromatic Leukodystrophy (arylsulfatase A), Multiple Sulfatase

Deficiency (arylsulfatase A, arylsulfatase B, arylsulfatase C), Sly Syndrome (β-glucuronidase), Sialidosis (a-Neuraminidase), I-cell Disease (UDP GlcNAc:lysosomal-enzyme N-acetyglucosamine-1-phosphotransferase), Pseudo-Hurler Polydistrophy (UDP GlcNAc:lysosomal-enzyme N-acetylglucosamine-1 -phosphotransferase), Mucolipidosis IV (mucolipin-1), α-Mannosidosis (α-mannosidase_, β-Mannosidosis (β-mannosidase), Fucosidosis (α-L-fucosidase), Aspartylglucosaminuria (N-aspartyl-β-glucosaminidase), Galactosialidosis (protective protein/cathepsin A or neuraminidase or β-galactosidase), Schindler Disease (α-N-acetyl-galactosaminidase), Cystinosis (cystine transport protein), Salla Disease (sialin), Infantile Sialic Acid Storage Disorder (sialin), Infantile Neuronal Ceroid Lipofuscinosis, Neuronal Ceroid Lipofuscinosis, (palmitoly-protein thioesterase), Prosaposin defects (either Saposin A, Saposin B, Prosaposin Saposin C or Saposin D domains of prosaposin).

Exemplary neurodegenerative diseases include, but are not limited to Parkinson's disease, Multiple Sclerosis, ALS, multi-system atrophy, Alzheimer's disease, stroke, progressive supranuclear palsy, fronto-temporal dementia with parkinsonism linked to chromosome 17 and Pick's disease.

The particles of the present invention may be administered to the subject per se or as part of a pharmaceutical composition. As used herein a “pharmaceutical composition” refers to a preparation of the particles encapsulating the active ingredients described herein with other chemical components such as physiologically suitable carriers and excipients. The composition may comprise particles which encapsulate just the lysosomal enzyme or just the small molecule agents. Alternatively, the composition may comprise particles which encapsulate both the lysosomal enzyme and the small molecule agents.

It will be appreciated that the small molecule agent may be co-formulated in the particles which comprise the lysosomal enzyme or the small molecule agent may be comprised in particles which do not comprise the lysosomal enzyme.

The purpose of the pharmaceutical composition is to facilitate administration of the active ingredients to the subject.

Herein the term “active ingredient” refers to the agents, which increase the amount or activity of the lysosomal enzymes in the brain.

Hereinafter, the phrases “physiologically acceptable carrier” and “pharmaceutically acceptable carrier” which may be interchangeably used refer to a carrier or a diluent that does not cause significant irritation to the subject and does not abrogate the biological activity and properties of the administered active ingredients. An adjuvant is included under these phrases.

Herein, the term “excipient” refers to an inert substance added to the pharmaceutical composition to further facilitate administration of an active ingredient of the present invention. Examples, without limitation, of excipients include calcium carbonate, calcium phosphate, various sugars and types of starch, cellulose derivatives, gelatin, vegetable oils and polyethylene glycols. The pharmaceutical composition may advantageously take the form of a foam or a gel.

Techniques for formulation and administration of drugs may be found in “Remington's Pharmaceutical Sciences,” Mack Publishing Co., Easton, Pa., latest edition, which is incorporated herein by reference.

The present invention contemplates administering the particles into the brain of the subject either directly, or indirectly via the blood brain barrier. Preferably, following administration at least 10% of the particles administered reach the brain, at least 20% of the particles administered reach the brain, at least 30% of the particles administered reach the brain, at least 40% of the particles administered reach the brain, at least 50% of the particles administered reach the brain, at least 60% of the particles administered reach the brain, at least 70% of the particles administered reach the brain, at least 80% of the particles administered reach the brain, at least 90% of the particles administered reach the brain or at least 95% of the particles administered reach the brain.

According to another embodiment, at least 50% of the particles administered reach the brain following 24 hours, at least 60% of the particles administered reach the brain following 24 hours, at least 70% of the particles administered reach the brain following 24 hours, at least 80% of the particles administered reach the brain following 24 hours, at least 90% of the particles administered reach the brain following 24 hours, at least 95% of the particles administered reach the brain following 24 hours.

According to another embodiment, at least 50% of the particles administered reach the brain following 48 hours, at least 60% of the particles administered reach the brain following 48 hours, at least 70% of the particles administered reach the brain following 48 hours, at least 80% of the particles administered reach the brain following 48 hours, at least 90% of the particles administered reach the brain following 48 hours, at least 95% of the particles administered reach the brain following 48 hours.

Suitable routes of administration include any of various suitable systemic and/or local routes of administration.

Suitable routes of administration may, for example, include the inhalation, oral, buccal, rectal, transmucosal, topical, transdermal, intradermal, transnasal, intestinal and/or parenteral routes; the intramuscular, subcutaneous and/or intramedullary injection routes; the intrathecal, direct intraventricular, intravenous, intraperitoneal, intranasal, and/or intraocular injection routes; and/or the route of direct injection into a brain of the subject.

The pharmaceutical composition may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer.

For transmucosal administration, penetrants appropriate to the barrier to be permeated are used in the formulation. Such penetrants are generally known in the art.

For oral administration, the pharmaceutical composition can be formulated readily by combining the active ingredients with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active ingredient doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration via the inhalation route, the active ingredients for use according to the present invention can be delivered in the form of an aerosol/spray presentation from a pressurized pack or a nebulizer with the use of a suitable propellant, e.g., a fluorochlorohydrocarbon such as dichlorodifluoromethane, trichlorofluoromethane, dichloro-tetrafluoroethane; carbon dioxide; or a volatile hydrocarbon such as butane, propane, isobutane, or mixtures thereof. In the case of a pressurized aerosol, the dosage unit may be determined by providing a valve to deliver a metered amount. Capsules and cartridges of, e.g., gelatin for use in a dispenser may be formulated containing a powder mix of the active ingredients and a suitable powder base such as lactose or starch.

The pharmaceutical composition may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

A pharmaceutical composition for parenteral administration may include an aqueous solution of the active ingredients in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredients may be in powder form for constitution with a suitable vehicle, e.g., sterile, pyrogen-free water based solution, before use.

The pharmaceutical composition may also be formulated in rectal compositions such as suppositories or retention enemas, using, e.g., conventional suppository bases such as cocoa butter or other glycerides.

The pharmaceutical composition should contain the active ingredients in an amount effective to achieve disease treatment.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays—e.g. lysosomal enzyme comprising particles may be tested for in-vitro activity in plasma or in other plasma mimicking environments. For example, a dose can be formulated in animal models (e.g. Fabry mice which comprise high levels of globotriaosylceramide) to achieve a desired brain concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

Dosage amount and interval may be adjusted individually to provide plasma or brain levels of the active ingredients which are sufficient to achieve the desired therapeutic effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of the composition to be administered will be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredients. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Ct. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### Preparation of the Nanocarriers

Nanoparticle clusters grafted with hyaluronan (GAGs) are prepared. These nanoparticles have the advantage of entrapping different therapeutic payloads and traffic them to different cell types in different administration routes. The human recombinant GCase is entrapped inside the GAGs. The recombinant enzyme can be obtained from Protalix; Shire or Genzyme. Following purification of the particles from non-entrapped protein using pronase, the structural properties of the GAGs-entrapped enzyme are analyzed by size distribution and surface charge, by a Malvern ZS Zetasizer, and shape by HR-SEM and Environmental SEM. Encapsulation efficiency is estimated by releasing the payload from the particles through disruption with hyaloronidase and deoxycholate, and determining the amount and activity of the entrapped enzyme. Activity is measured in vitro at lysosomal pH (5.5) by following the hydrolysis of 4-methyl umbeliferyl glucocpyramoside to 4-methyl umbeliferon, which fluoresces at 640 ηm. These measurements will allow us to determine the specific activity of the entrapped enzyme. The specific activity of the entrapped enzyme can be compared to that of the non-entrapped recombinant enzyme.

Time dependent stability in human serum and enzyme release from the nanocarriers is tested in human serum at 37° C. This stability is compared to non-entrapped enzyme, as it is possible that the entrapped enzyme will be stabilized by the nanocarriers.

Exemplary protocol for preparation of Ambroxol-loaded Gagomers:
Lipid ratio: 1:10 (mole/mole) DLPG:DLPE
HA/lipid ratio: 1:10 (w/w) HA:PE
Drug/lipid ratio 1:12 (mole/mole) Ambroxol: PE (total lipids)
Final lipid concentration: 6 mg/ml

Preparation of activated HA:
1. Weigh 10 mg HA (700,000 Da Lifecore) in a scintillation vial and dissolve in 5 ml MES buffer 100 mM pH=5.5 (2 mg/ml) for ˜2 hours while stirring at room temperature.
Immediately before use: prepare 1M stocks of sulfo-NHS and EDC. Weigh 11.2 mg EDC and 12.3 mg of sulfo-NHS and dissolve each in 40 μl MES buffer 100 mM pH=5.5 Vortex for a few seconds until the powder dissolves. Before weighing, take the EDC out from the −20 and the sulfo-NHS from 4 degrees and bring to room temperature.
2. Add to the HA solution 33.5 μl of sulfo-NHS and immediately afterwards 33.5 μl of the EDC while stirring. Continue stirring for 15 minutes at room temperature. The activation can continue up to 30 minutes.
3. Add 4.62 ml of the activated HA to the lipid suspension.

Preparation of the lipid suspension:
1. Weigh (after bringing the lipids to room temperature—takes about 20 minutes) 82.6 mg of DLPE and 10 mg of DLPG and put it in a 250 ml colba. All lipids are stored at −20° C.
2. Add 15 ml 96% Ethanol.
3. Heat to 60° C. and stir until a clear solution is received.
4. Mix and evaporate in Buchi evaporator for a total of 1 hour (water bath temp: 65° C., rotation speed: 4).
5. After evaporation use nitrogen gas pistol to evaporate leftovers.
6. Add a total of 10.8 ml MES buffer 0.1M pH5.5 and the Ambroxol to the dry film. Heat to 60° C. and vortex for several minutes in order to recover all the material from the colba walls. Shake for 2 hours at 60° C.
7. Add 4.62 ml activated HA.
8. Shake over night at room temperature.

Washings:
1. Wash the lipids from excess HA and cross linkers by-products by ultracentrifugation: 80,000 RPM 45 minutes, 4° C.:
   a) Put 1.75 ml of the GAG suspension in each tube and add 1.75 ml of HBS: Hepes 20mM, NaCl 150 mM pH8.2. Total of 3.5 ml in each tube. Spin down in the terms written above.
   b) Discard the supernatant and save it for absorption. Re-suspend the pellet in 1 ml HBS pH8.2 and afterwards add 1 ml HBS pH8.2. Spin down.
   c) Discard the supernatant and save it for absorption. Re-suspend the pellet in 1 ml HBS pH7.4 and afterwards add 1 ml HBS pH7.4. Spin down.
   d) Discard the supernatant and put 1.75 ml of HBS pH7.4 and resuspend the pellet in order to achieve the original lipid concentration and salinity.

Lvophilization:
1. Sonicate 10 min in bath sonicator in small batches (maximum 2 ml each time).
2. Distribute 0.5 ml of the suspension to lyophilization vials.
3. Freeze at −80° C. for at least 2 hours.
4. Lyophilize vials for 48 hours.
5. Lyophylized gagomers should be rehydrated with the original volume of DDW or medium.
6. Heat at 37° C. for 20 min and sonicate Gagomers in bath sonicator before use.

### Example 2

### Testing the Nanocarriers in vitro

Following preparation and in vitro testing of the entrapment efficacy, the internalization mechanism of the encapsulated protein may be tested. To this end, fluorescently labeled particles and cells with fluorescently labeled membranes are employed (they will be prepared by including Alexa 488-HA on the GAGs' surface and by a CellTracker DilC18(5)-DS, which labels the membranes of cells). Using a combination of bio-AFM and spinning disc confocal microscopy it is possible to visualize the internalization process. This will enable following the interaction between the particles and the receiving cell membrane and the mode of internalization.

To test whether entry is clathrin dependent or non-clathrin dependent different drugs may be used. To block clathrin dependent endocytosis dynosaure, sucrose or monodensylcadaverine (MDC) are used. To block non-clathrin dependent endoxytosis genistein and nystatin are used. The readout is intracellular localization of the recombinant enzyme by interacting the cells, after fixation, with a specific antibody and visualizing it using a fluorescently labelled secondary antibody. This enables tracing of the intracellular localization of the enzyme, which is of prime importance since the enzyme has to reach the lysosomes in order to be active. Enzymatic activity is tested in the cells by preparing a cell lysate and employing the in vitro assay described above.

The ability of the entrapped recombinant GCase to reach lysosomes of a wide variety of tissue culture cells is tested by adding it to the cell culture media. Exemplary cells include human endothelial cells (HUVEC, human umbilical derived vascular endothelial cells), dopaminergic cells (SH-SY5Y), and human monocytes (THP-1), which are the cells most severely affected in GD patients. The THP-1 cells may be treated with PMA in order to mature them into macrophages. Enzymatic activity is the readout, and the assay is performed 6-48 hours after addition of the nanoparticles, as determined by the protein release assay.

### Example 3

### Ability of the Enzyme to Cross the Bbb and to Function in the Brain

The ability of Cy5-labeled enzyme (which is prepared using the Amersham labelling kit), entrapped in the GAGs, to reach rat brain parenchyma upon intranasal administration is investigated. GAGs is sprayed intranasaly and 4-6 h later, perfusion is performed to purify brain parenchyma from excessive (free) enzyme. The animals (rats) are sacrificed and thin sections (5-8 μm) of frozen brain are prepared for immunohistochemistry, tracking the presence of the labelled enzyme, using confocal microscopy. Since the background fluorescence in brain tissue is green at excitation of 488 ηm, the Alexa dye 555 (which will provide a red fluorescence) may be used as the marker. Staining can be against with CD11b/CD45/CD68 for microglia and Neu for neurons. Control rats may receive empty vector (GAGs) and GAGs with Cy5-labeled BSA. The ability of the enzyme to reach and function in the brain of rats is tested, by assaying its activity in lysates prepared from the treated brains.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A composition comprising particles which encapsulate an agent selected from the group consisting of a lysosomal enzyme, a small molecule which lowers an amount of a substrate of a lysosomal enzyme in a lysosome of a cell and a combination thereof.

2. A method of treating a neurodegenerative disorder, comprising administering to a subject in need thereof a therapeutically effective amount of an agent selected from the group consisting of a lysosomal enzyme, a small molecule which lowers an amount of a substrate of a lysosomal enzyme in brain cells of the subject and a combination thereof, wherein said lysosomal enzyme and said small molecule are encapsulated within particles, thereby treating the neurodegenerative disorder.

3-5. (canceled)

6. The method of claim 2, wherein said administering comprises intranasally administering.

7. (canceled)

8. The method of claim 2, wherein said particles have a charged external surface.

9. The method of claim 2, wherein said particles comprise a neutral external surface.

10. The method of claim 2, wherein said particles comprise lipids.

11. The method of claim 10, wherein said particles comprise cationic lipids.

12. The method of claim 11, wherein said cationic lipid is selected from the group consisting of 1,2-Dilauroyl-sn-Glicero-3-Phosphoethanolamine (DLPE) and 1,2-Dilauroyl-sn-Glicero-3-Glycerol (DLPG), dioleoyl-1,2-diacyl-3-trimethylammonium-propane (DOTAP, at 18:1; 14:0; 16:0, 18:0) and N-[1-(2,3-dioleyloxy)propyl]-N,N,N-trimethlylammonium chloride (DOTMA); dimethyldioctadecylammonium (DDAB); 1,2-dilauroyl-sn-glycero-3-ethylphosphocholine (Ethyl PC, at 12:0; 14:0; 16:0; 18:0; 18:1; 16:0-18:1); 1,2-di-(9Z-octadecenoyl)-3-dimethylammonium-propane and 3β-[N-(N′,N′-dimethylaminoethane)-carbamoyl]cholesterol hydrochloride (DC-Cholesterol).

13. The method of claim 10, wherein said lipids comprise a neutral lipid.

14. The method of claim 13, wherein said neutral lipid comprises phosphatidylethanolamine or dioleilphosphatidylethanolamine (DOPE).

15. The method of claim 10, wherein said lipids comprise anionic phospholipids.

16. The method of claim 15, wherein said anionic phospholipids are selected from the group consisting of phosphatidylserine, phosphatidic acid, phosphatidylcholine and phosphatidyl glycerol.

17. The method of claim 2, wherein a targeting moiety is attached to an outer surface of said particles.

18. (canceled)

19. The method of claim 17, wherein said targeting moiety comprises a glycosaminoglycan.

20. (canceled)

21. The method of claim 2, wherein said glycosaminoglycan comprises HA.

22. (canceled)

23. The method of claim 2, wherein said neurodegenerative disorder is Parkinson's.

24. The method of claim 2, wherein said neurodegenerative disorder comprises a neurometabolic disorder.

25-26. (canceled)

27. The method of claim 2, wherein said lysosomal enzyme is GCase.

28. (canceled)

29. The composition of matter or method of claim 2, wherein said small molecule increases an activity and/or amount of a lysosomal enzyme in brain cells of the subject.

30. The method of claim 2, wherein said small molecule enhances a passage of a mutant lysosomal enzyme from the endoplasmic reticulum to the lysosome of brain cells of the subject.

31. The method of claim 2, wherein said small molecule is co-formulated in said particles which comprise said lysosomal enzyme.

32. The method of claim 2, wherein said small molecule is comprised in particles which do not comprise said lysosomal enzyme.

33. The method of claim 2, wherein said lysosomal enzyme is GCase and said small molecule binds GCase.

34. The method of claim 2, wherein said lysosomal enzyme is Gcase and said small molecule comprises a glucosyl-ceramide synthase inhibitor.

35. The method of claim 2, wherein said lysosomal enzyme is GCase and said small molecule is Ambroxol.

36. A pharmaceutical composition comprising the composition of claim 1.
